(19) United States
(12) Patent Application Publication
(10) Pub. No.: US 2019/0351203 A1
(43) Pub. Date: Nov. 21, 2019

(54) Composition Applicator with Integrated Light Source

(71) Applicant: ENTROTECH LIFE SCIENCES , INC., San Francisco, CA (US)

(72) Inventor: James E. McGuire, JR., Palm Beach, FL (US)

(73) Assignee: entrotech life sciences, inc., San Francisco, CA (US)

(21) Appl. No.: 16/483,002

(22) PCT Filed: Feb. 8, 2018
(86) PCT No.: PCT/US2018/017473

§ 371 Date: Aug. 1, 2019

Related U.S. Application Data
(60) Provisional application No. 62/461,601, filed on Feb. 21, 2017.

(51) Int. Cl. A61M 35/00; B05C 9/14

(52) U.S. Cl. CPC A61M 35/003; B05C 17/002; B05C 9/14

(57) ABSTRACT

An apparatus for application of a composition to a surface, the apparatus comprising: receptacle(s) for all component(s) of the composition in one or multiple parts; an applicator pad configured for dispensing the composition onto the surface; and at least one integrated light source operable to illuminate the surface while the surface is contacted with the applicator pad. Methods for using the apparatus include a method for disinfecting a skin surface wherein the composition is an antimicrobial skin prep solution comprising at least one fluorescing agent and the at least one integrated light source operates to visually evaluate coverage of the skin prep solution

### CROSS-REFERENCE TO RELATED APPLICATIONS

This application claims priority from U.S. Provisional Patent Application No. 62/461,601, filed on Feb. 21, 2017, which is incorporated by reference herein in its entirety.

### BACKGROUND OF THE INVENTION

The present invention relates generally to apparatus for application of a composition to a surface, including antimicrobial skin prep solutions applied to skin surfaces.

A variety of compositions can be applied to a variety of surfaces. For example, antimicrobial skin prep solutions are often applied to patient's skin before medical procedures. 3M's DURAPREP Surgical Solution (an iodine povacrylex and isopropyl alcohol solution available from 3M Company of St. Paul, Minn.) is a well-known skin prep solution. As a further example, CHLORAPREP (2% chlorhexidine gluconate based on total weight of a solution containing 70% isopropyl alcohol and 30% water available from Becton, Dickinson and Company of Franklin Lakes, N.J.) is also a well-known skin prep solution.

Recognizing the importance of being able to visualize where a skin prep solution has been applied to a skin surface, in order to ensure sufficient coverage amongst other potential reasons, it is known to include visual indicators within skin prep solutions. For example, CHLORAPREP skin prep solutions are available from Becton, Dickinson and Company with a HI-LITE ORANGE or SCRUB TEAL tint to facilitate site visualization. The inclusion of such tint, however, leads to an unsightly appearance on the skin before and continuing until after its use is no longer necessary.

Alternatives to unsightly compositions are known. For example, U.S. Pat. No. 3,640,889 describes a fluorescent skin-marking composition useful for marking skin prior to medical procedures. The composition is invisible in visible light, but it fluoresces under ultraviolet (UV) light.

As another example, U.S. Pat. No. 4,572,831 and its divisional, U.S. Pat. No. 4,610,806, describe fluorescamine compounds that react with the epidermis when applied to the skin and form, within about fifteen to thirty minutes after application to the skin, a long-lasting reaction product, which is invisible in ordinary light but which fluoresces intensely under UV light. Organic solutions of such compounds, preferably containing a fugitive dyestuff or pigment and used as the marking fluid of a conventional felt-tipped pen, can be used to mark skin for radiological or diagnostic purposes.

PCT Patent Publication No. WO 1998/020094 describes compositions for disinfection of skin and mucous membranes that contain an optical brightener. The compositions exhibit intense fluorescence in the visible wavelength range on exposure to UV light and, thus, permit monitoring of the treated skin or mucous membrane surfaces for ensuring complete disinfection, but without exhibiting the disadvantages associated with the use of conventional dyes, such as discoloration of the skin and articles.

Further, PCT Patent Publication No. WO 2007/133532 describes a process for the preparation of an aqueous ready-to-use disinfectant composition comprising at least one peracid and at least one organic compound capable of showing fluorescence on UV irradiation.

While these alternatives provide advantages, they lack efficiency in their use. More efficient and adequate methods and associated apparatus for application of compositions to surfaces, such as in the application of a skin prep solution to the skin, are desirable.

### SUMMARY OF THE INVENTION

Improved apparatus of the invention facilitate efficient and adequate application of a composition to a surface. The apparatus comprises: receptacle(s) for all component(s) of the composition in one or multiple parts; an applicator pad configured for dispensing the composition onto the surface; and at least one integrated light source operable to illuminate the surface while the surface is contacted with the applicator pad.

Advantageously, applicators of the invention include at least one integrated light source. The integrated light source facilitates efficient and visual observation of a composition's coverage or lack thereof on a surface, where the composition is applied from the applicator. The visual observation allows for evaluation of, for example, existence and/or adequacy of coverage on the surface.

Methods for using the apparatus include a method for disinfecting a skin surface wherein the composition is, for example, an antimicrobial skin prep solution comprising at least one fluorescing agent and the light source operates to visually evaluate coverage of the solution on the skin surface.

### BRIEF DESCRIPTION OF THE SEVERAL VIEWS OF THE DRAWINGS

FIG. 1 is a perspective view of an exemplary skin prep solution applicator of the invention.

FIG. 2 is a side perspective view of the exemplary skin prep solution applicator of FIG. 1.

FIG. 3 is a cut-away perspective view of the exemplary skin prep solution applicator of FIG. 1.

FIG. 4 is an exploded perspective view of the exemplary skin prep solution applicator of FIG. 1.

FIG. 5A is a cut-away side perspective view of the exemplary skin prep solution applicator of FIG. 1.

FIG. 5B is a partial cut-away side perspective view of a portion of the exemplary skin prep solution applicator of FIG. 5A, taken proximate the thumb rest thereon.

FIG. 6A is a side perspective view of the exemplary skin prep solution applicator of FIG. 1, as viewed from a side of the applicator opposite that of FIG. 2.

FIG. 6B is a cut-away side perspective view of a portion of the exemplary skin prep solution applicator of FIG. 6A, taken proximate the breaking rib thereon.

FIG. 6C is a side perspective view of a portion of the exemplary skin prep solution applicator of FIG. 6A, taken proximate the handle hinge thereon.

FIG. 6D is a side perspective view of a portion of the exemplary skin prep solution applicator of FIG. 6A, taken proximate the breaking rib thereon.

FIG. 7A is a rearwardly oriented side perspective view of the exemplary skin prep solution application of FIG. 1.

FIG. 7B is an upwardly oriented side perspective view of a portion of the exemplary skin prep solution applicator of FIG. 7A, taken proximate the light source.

FIG. 7C is a partial cut-away side perspective view of the portion of the exemplary skin prep solution applicator of FIG. 7B.

FIG. 8A is a partial perspective view of the exemplary skin prep solution applicator of FIG. 1, with an exploded view of components of the light source therein.

FIG. 8B is partial cut-away side perspective view of a portion of the exemplary skin prep solution applicator of FIG. 8A, taken proximate the assembled light source therein.

### DETAILED DESCRIPTION OF THE INVENTION

Composition applicators of the invention include at least one integrated light source. With respect to the term “integrated” as used in conjunction with “light source,” it is to be understood that, during application of a composition from the applicator, the light source is not discrete from the remainder of the applicator of which it is a part. While individual components of a light assembly (e.g., light bulb, light bulb cover, battery, battery compartment lid/cap, and the like) may be readily separable from the applicator, as in the case where replacement of such an individual component is needed, all individual components necessary for illumination of the light source are included within the applicator during its use. By utilizing at least one light source integrated with the applicator, coverage of the composition on the surface can be evaluated essentially simultaneously with or shortly after application of the composition on the surface. Further, separate handling of a light source from the composition applicator is not necessary. Thus, inefficiencies associated with distinct applicators and light sources, for example, those needed to illuminate fluorescing agents within the composition, are eliminated according to the invention.

According to one aspect of the invention, the applicator comprises receptacle(s) for all components of the composition in one or multiple parts. In an exemplary embodiment, the applicator facilitates application of a skin prep solution onto a skin surface. In order to dispense the skin prep solution therein, any suitable mechanisms can be utilized. For example, when application of the skin prep solution is desired, any suitable mechanism is utilized to initiate dispensing of the skin prep solution from within the applicator, combining multiple parts thereof, as necessary.

In one embodiment, at least all major component(s) of the skin prep solution are contained within an appropriately sized housing until the skin prep solution is applied to a skin or other surface. As used herein with reference to component(s), the term “major” is to be understood as used in relation to volume of the composition. Thus, “major component(s)” of the skin prep solution are those component(s) which, altogether, comprise more than fifty-percent of the volume of the skin prep solution. The housing may be one with (i.e., not readily and reversibly separable from) the applicator or discrete from (i.e., initially, but readily and, preferably, reversibly one with) the applicator. If the latter, the housing is inserted into the applicator prior to dispensing of the skin prep solution therein. Any suitable housing can be utilized, with the housing having one or multiple distinct compartments for storage of the one or multiple parts of the composition before they are dispensed within the applicator. In a preferred embodiment, the housing takes a form similar to an ampoule. In an exemplary embodiment, the housing takes a form similar to an ampoule having a capacity of about 26 milliliters.

Applicators and methods of the invention are advantageously used with a variety of compositions, not all of which contain a fluorescing agent. In a preferred embodiment, however, at least one fluorescing agent is included as a component of the composition. For example, a fluorescing agent can be applied to the skin or other surface as a component of the skin prep solution. Any suitable fluorescing agent can be used. For example, optical brighteners are described in PCT Patent Publication No. WO 1998/020094 and can be used as fluorescing agents in the present invention. One commercially available fluorescing agent suitably used in the present invention is that fluorescent whitening agent, also referred to as an optical brightener, of the thiophenediyl benzoxazole class available from BASF Schweiz AG (Switzerland) under the trade designation, TINOPAL OB.

The fluorescing agent(s) can be contained within the housing, or they can be mixed with the major component(s) of the skin prep solution after they are dispensed within the housing. For example, a fluorescing agent can be included within a filter through which the major component(s) of the skin prep solution dispensed from the housing travel within the applicator before application of the solution to skin or another surface using, for example, an applicator pad.

Any suitable applicator pad can be used. In an exemplary embodiment, the applicator pad comprises foam. A variety of suitable foam and other materials are known to those of ordinary skill in the art. The invention is not limited with respect thereto.

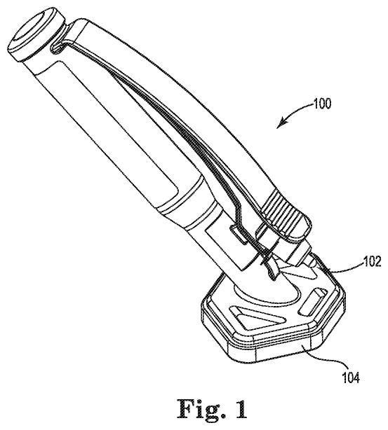

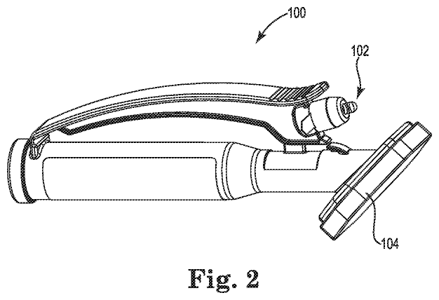

An exemplary skin prep solution applicator 100 of the invention is illustrated in the perspective views of FIGS. 1-2. As illustrated therein, the applicator 100 has an integrated light source 102 and applicator pad 104 to facilitate efficient and adequate application of a skin prep solution (not shown) to a surface.

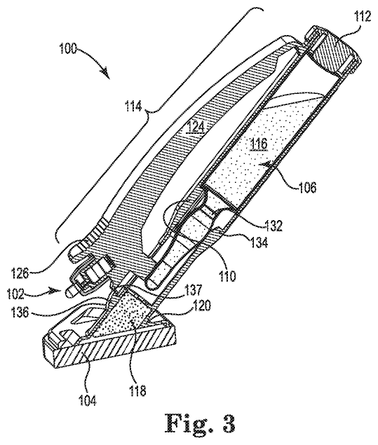

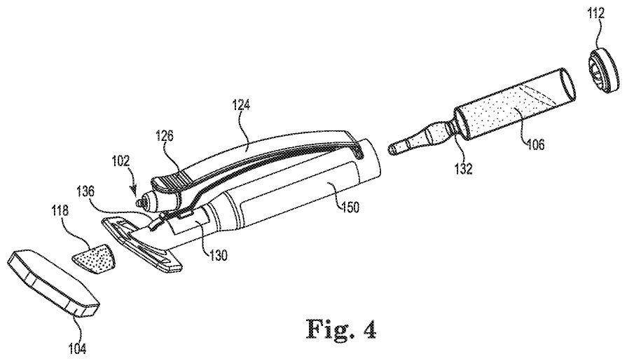

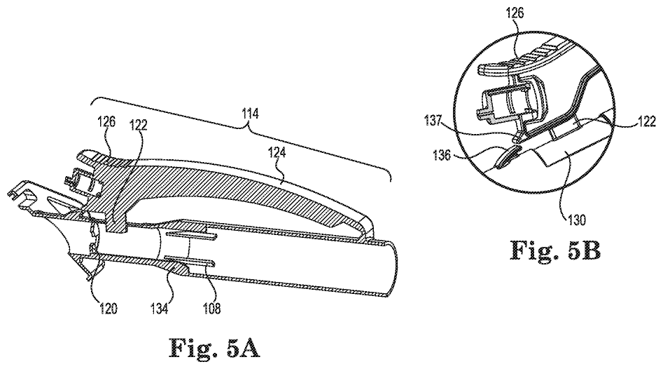

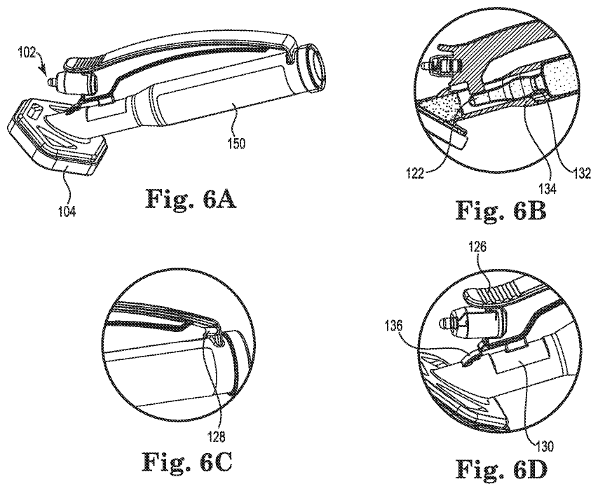

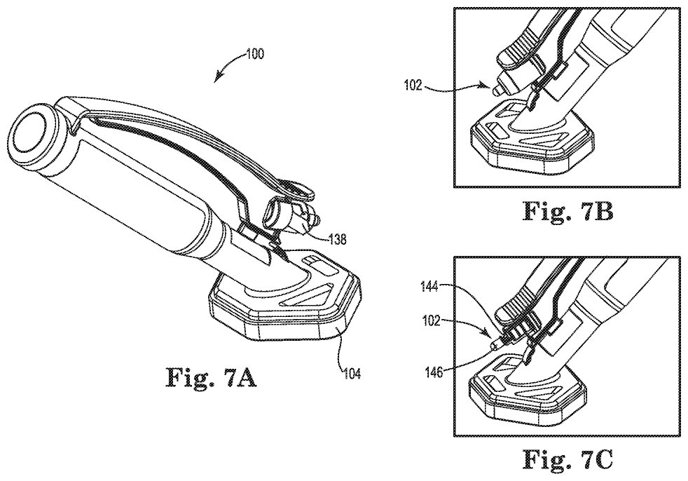

Further details of the exemplary skin prep solution applicator 100 are illustrated in the views of FIGS. 3-8B. A housing 106 in the form of an ampoule is sandwiched between one or more molded-in retaining ribs 108 in cooperation with stepped internal surfaces 110 and a cap 112 within the applicator 100. The housing 106 contains the major component(s) of the skin prep solution 116. In the embodiment illustrated, the skin prep solution component(s) 116 of the housing 106 flow through a filter 118 before being applied to a surface using the applicator pad 104 of the applicator 100. A containment wall 120 facilitates secure placement of the filter 118 within the applicator 100.

To initiate dispensing of the skin prep solution within the applicator 100, a breaking rib 122 or the like is utilized on a crush lever 124 of a handle 114 to break the housing 106 by, for example, creating an opening in a wall of the housing 106 via application of mechanical force to a thumb rest 126 or similar feature of the crush lever 124. The handle 114 containing the crush lever 124 is affixed to a distal end of the applicator 100 by, for example, a hinge 128. The thumb rest 126 is positioned on the opposite end of the handle 114. Mechanical force applied to the thumb rest 126 to break the housing 106 is transmitted through a deformable area 130 (e.g., thin-walled portion) of the applicator 100 to a break notch 132 on the housing 106 via the breaking rib 122. The amount of mechanical force required to break the housing can be adjusted by, for example, adjusting the distance between a fulcrum 134 positioned between the break notch 132 of the housing 106 and a tip of the breaking rib 122 on the crush lever 124 of the handle 114.

In a further embodiment, once the thumb rest 126 is depressed to break the housing 106, it remains in the depressed position utilizing a catch 136 on the applicator 100 for receipt of a reciprocal catch 137 on the handle 114. Although not shown in the depressed position in FIGS. 1-8B, the respective catches 136, 137 are illustrated therein.

Any suitable light source 102 can be used in applicators 100 of the invention. Exemplary light sources 102 include those emitting visible and/or ultraviolet light. In a preferred embodiment, the light source 102 comprises a light emitting diode (LED). Exemplary LEDs emit light having a wavelength of about 10 nanometers to about 430 nanometers and with light spreading at an angle of about twenty degrees to about sixty degrees—i.e., the “light spread” of the LED. In a preferred embodiment, a LED emits light having a wavelength of about 400 nanometers to about 430 nanometers and with light spreading at an angle of about thirty degrees to about sixty degrees. A preferred LED emits light having a wavelength of about 405 nanometers with an approximately 60-degree light spread. Orientation of the light source 102 on the applicator 100, and thus orientation of light from the light source 102 on the surface onto which the composition is applied during use of the applicator 100, can be fixed or flexible to allow for adjustment of the light source 102 to adequately illuminate a desired area. In an exemplary embodiment, as illustrated in FIGS. 1-8B, the light source 102 is integrated with the handle 114 of the applicator 100. However, recognize that the light source 102 can be integrated with other portions of the applicator 100 in other embodiments.

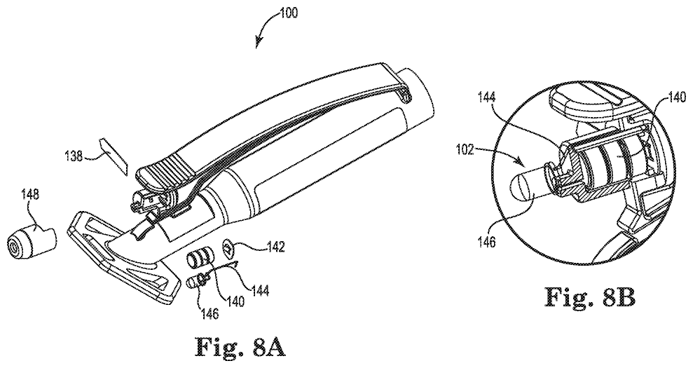

In order to activate the light source 102, any suitable mechanism can be utilized. In one embodiment, a light assembly used in applicators 100 of the invention comprises a pull-out tab 138, which is utilized to close an electrical circuit that activates the light source 102. For example, as illustrated in FIG. 8B, the pull-out tab 138 can, when pulled out, close an electrical circuit formed using one or more batteries 140 otherwise in physical and electrical contact, by way of assistance of a spring contact 142 or the like, with one or more legs 144 of a light bulb 146. In the illustrated embodiment, these components of the light assembly are contained within the handle 114 using a cover 148, such as the snap-on cover 148 illustrated therein.

Any suitable material and configuration can be used for the pull-out tab 138. A variety of pull-out tabs 138 are known and include materials such as plastic, paper, and the like. Any suitable electrical source can be used to illuminate the light source 102. In the exemplified embodiment, three A76 alkaline batteries 140 with an expected life of about two hours are used.

The applicator 100 may contain a product label 150. Any suitable information may be included on such a product label 150 as known to those of ordinary skill in the art. For example, instructions for applying contents of the applicator 100 onto a surface and viewing coverage of the skin prep solution on a surface utilizing the light source 102 may be included on such a product label 150.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited unless expressly stated otherwise. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

As used throughout, ranges may be used as shorthand for describing each and every value that is within the range. Any value within the range can be selected as the terminus of the range. Similarly, any discrete value within the range can be selected as the minimum or maximum value recited in describing and claiming features of the invention. Further, any properties described or measured herein are those existing at room temperature and atmospheric pressure unless specified otherwise.

1. An apparatus for application of a composition to a surface, the apparatus comprising:
one or more receptacles for all components of the composition in one or multiple parts;
an applicator pad configured for dispensing the composition onto the surface; and
at least one integrated light source operable to illuminate the surface while the surface is contacted with the applicator pad.

2. The apparatus of claim 1, wherein the apparatus is for application of a skin prep solution to a skin surface.

3. The apparatus of claim 1, wherein at least one fluorescing agent is included as one of the components of the composition.

4. The apparatus of claim 3, wherein the at least one fluorescing agent is included within a filter through which major component(s) of the composition travel within the applicator before application of the composition to the surface via the applicator pad.

5. The apparatus of claim 1, wherein at least all major component(s) of the composition are contained within an appropriately sized housing until being dispensed therefrom for application of the composition to the surface.

6. The apparatus of claim 5, wherein the housing is discrete from the applicator until use of the apparatus for applying the composition onto the surface.

7. The apparatus of claim 5, wherein the housing takes the form of an ampoule.

8. The applicator of claim 7, wherein the housing comprises a break notch.

9. The applicator of claim 8, wherein the applicator comprises a breaking rib operable by application of mechanical force to a crush lever to break the housing in order to dispense the components therein for application of the composition to the surface.

10. The applicator of claim 1, wherein the composition is contained in multiple parts.

11. The applicator of claim 1, wherein the at least one integrated light source comprises an ultraviolet light source.

12. The applicator of claim 1, wherein the at least one integrated light source comprises a light emitting diode.

13. The applicator of claim 1, further comprising a pull-tab for operation of the at least one integrated light source.

14. The applicator of claim 1, wherein the at least one integrated light source is operable to emit light having a wavelength of about 400 nanometers to about 430 nanometers and with light spreading on the surface at an angle of about thirty degrees to about sixty degrees.

15. The applicator of claim 1, wherein the at least one integrated light source is operable to emit light having a wavelength of about 405 nanometers with an approximately 60-degree light spread on the surface.

16. A method of utilizing the apparatus of claim 1, comprising:
if initially discrete from the applicator, inserting a housing containing at least all major component(s) of the composition into the applicator;
dispensing the composition within the applicator;
contacting the applicator pad of the applicator with the surface to apply the composition to the surface;
activating the at least one integrated light source to evaluate coverage of the composition on the surface essentially simultaneously with or shortly after application of the composition to the surface.

17. The method of claim 16, wherein the composition comprises at least one fluorescing agent contained within the housing.

18. The method of claim 16, wherein at least one fluorescing agent is mixed with the major component(s) of the composition after they are dispensed from the housing and before the composition is applied to the surface from the applicator.

19. A method of disinfecting a skin surface, comprising steps of:
applying an antimicrobial skin prep solution from the applicator of claim 1 to the skin surface to provide a disinfected skin surface;
activating the at least one light source on the applicator to visually evaluate coverage of the skin prep solution on the surface; and
optionally, after visually evaluating coverage of the skin prep solution on the surface, applying additional skin prep solution from the applicator to the surface.

20. The method of claim 19, wherein the skin prep solution comprises at least one fluorescing agent.
